(19) United States
(12) Patent Application Publication
(10) Pub. No.: US 2014/0005447 A1
(43) Pub. Date: Jan. 2, 2014

(54) Method for Production of 1-Chloro-3,3,3-Trifluoropropene

(71) Applicant: Central Glass Company, Limited, Ube-shi (JP)

(72) Inventors: Masamune OKAMOTO, Fujimino-shi (JP); Hideaki IMURA, Iruma-gun (JP); Naoto TAKADA, Iruma-gun (JP)

(73) Assignee: Central Glass Company, Limited, Ube-shi (JP)

(21) Appl. No.: 13/929,219

(22) Filed: Jun. 27, 2013

(30) Foreign Application Priority Data
Jun. 29, 2012 (JP) 2012-146500
Jun. 25, 2013 (JP) 2013-133057

(51) Int. Cl. C07C 17/20; C07C 17/23

(52) U.S. Cl. CPC C07C 17/208; C07C 17/23
USPC 570/156; 570/160

(57) ABSTRACT

A production method of 1-chloro-3,3,3-trifluoropropene according to the present invention includes bringing a raw material composition containing 1,3,3,3-tetrafluoropropene and an acid composition containing hydrogen chloride into contact with each other in gas phase in the presence of a catalyst. This production method allows not only use of hydrogen chloride containing hydrogen fluoride, which has been generated during a preceding step (production of 1,1,1,3,3-pentafluoropropane as a raw material or analogues thereof), but also use of any of trans and cis isomers of the 1,1,1,3,3-pentafluoropropane for production of the 1-chloro-3,3,3-trifluoropropene. It is thus possible to efficiently produce the 1-chloro-3,3,3-trifluoropropene, which is known as an environment-adaptive chlorofluorocarbon. As the catalyst, preferred is an alumina catalyst treated by contact with hydrogen fluoride.

### FIELD OF THE INVENTION

The present invention relates to a method for production of 1-chloro-3,3,3-trifluoropropene, which is useful as a solvent, a cleaning agent, a coolant, a working fluid, a propellant, a raw material for production of fluorine resins etc.

### BACKGROUND OF THE INVENTION

It is known that 1-chloro-3,3,3-trifluoropropene is a next-generation environment-adaptive chlorofluorocarbon that can be easily decomposed in the air and does not cause depletion of the ozone layer. Herein, 1-chloro-3,3,3-trifluoropropene has a double bond in the molecule and exists as trans and cis geometric isomers. The trans and cis isomers of 1-chloro-3,3,3-trifluoropropene are hereinafter sometimes called “1233E” and “1233Z”, respectively, by their identification numbers with additional symbols. The 1-chloro-3,3,3-trifluoropropene is simply called “1233” in the case where there is no need to distinguish the trans and cis isomers or in the case where it refers to a mixture of the trans and cis isomers. The trans isomer 1233E has been put into practical use as a blowing agent etc., whereas the cis isomer 1233Z has been put into practical use as a solvent etc. For example, Patent Document 1 discloses a blowing agent containing 1233E. Patent Document 2 discloses a method for purifying 1233Z. Patent Document 3 discloses a method for producing 1233 by contact of 1,1,1,3,3-pentafluoropropane (sometimes called “245”) and hydrogen chloride in gas phase. Patent Document 4 discloses a method for synthesizing 245, as a raw material for production of 1233, by reaction of 1,1,1,3,3-pentachloropropane (sometimes called “240”) with hydrogen fluoride.

Further, 1,3,3,3-tetrafluoropropene also has a double bond in the molecule and exists as trans and cis geometric isomers. The trans and cis isomers of 1,3,3,3-tetrafluoropropene are hereinafter sometimes called “1234E” and “1234Z”, respectively. The 1,3,3,3-tetrafluoropropene is simply called “1234” in the case where there is no need to distinguish the trans and cis isomers or in the case where it refers to a mixture of the trans and cis isomers. The trans isomer 1234E (boiling point: −19° C.) has been put into use as a coolant in a refrigerator, an automotive air conditioner etc., whereas the cis isomer 1234Z (boiling point: 9° C.) has been put into use as a working fluid in a high-temperature heat pump, a raw material for production of fluorinated propyne (CF₃—C≡CH) etc. The fluorinated propyne is useful as a resin blowing agent, a heat transfer fluid or a propellant. Patent Document 5 discloses a method for producing 1234 by dehydrofluorination of 1,1,1,3,3-pentafluoropropane (sometimes called “245”) in gas phase in the presence of a zirconium compound-carrying catalyst having a zirconium compound carried on a metal oxide or activated carbon.

As indicated in the following scheme, 1,3,3,3-tetrafluoropropene (1234) can be produced by gas-phase thermal decomposition and dehydrofluorination of 1,1,1,3,3-pentafluoropropane (245). There is however no reaction process that enables selective production of either trans isomer (1234E) or cis isomer (1234Z) of 1,3,3,3-tetrafluoropropene. The reaction product is obtained in the form of a mixture of trans isomer (1234E) and cis isomer (1234Z). The generation ratio of these trans and cis isomers is varied depending on the thermodynamic equilibrium.

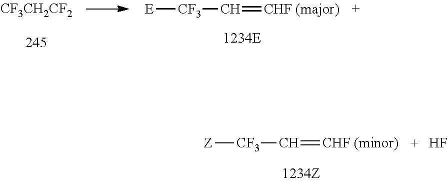

By calculation of a Boltzmann distribution of the trans isomer (1234E) and cis isomer (1234Z) using a functional B3LYP/6-311+G**, which is regarded as compatible with the experimental results of the above reaction, it has become apparent that the generation ratio of the trans isomer (1234E) increase with temperature as shown in FIG. 1. Even when the gas-phase dehydrofluorination of the 245 is performed in the presence of the catalyst, however, the reaction rate is low under temperature conditions of 200° C. or lower. There thus arises a possibility of decomposition/carbonization of the raw material or reaction product, serious corrosion of the reaction vessel etc. under high-temperature conditions. It is difficult to arbitrarily control the generation ratio of the trans and cis isomers of the 1234 and thereby difficult to produce only either one of the trans isomer (1234E) and cis isomer (1234Z) from the 245. In addition, the isomer generation ratio of the reaction product (1234) does not always become as desired by a manufacturer.

On the other hand, 1,1,1,3,3-pentafluoropropane (245) can be industrially produced by fluorination reaction of an organic compound such as 1,1,1,3,3-pentachloropropane (240) with hydrogen fluoride as indicated in the following scheme. In this reaction, hydrogen chloride (HCl) containing unreacted hydrogen fluoride (HF) is generated as a by-product.

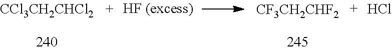

In order to remove the unreacted hydrogen fluoride and obtain the hydrogen chloride at a high purity, there is a need to separate the hydrogen fluoride by the use of a pressure distillation column. In view of the cost performance of such distillation operation, it is often the case that the hydrogen chloride containing the hydrogen fluoride is disposed of as a waste after neutralization treatment.

### PRIOR ART DOCUMENTS

Patent Document 1: Published Japanese Translation of International Patent Publication No. 2011-504538
Patent Document 2: Japanese Laid-Open Patent Publication No. 2010-202640
Patent Document 3: Japanese Laid-Open Patent Publication No. 2010-64990
Patent Document 4: International Patent Publication No. 2001/036355
Patent Document 5: Japanese Laid-Open Patent Publication No. 2008-19243

### SUMMARY OF THE INVENTION

It is an object of the present invention to provide a method for efficient production of 1-chloro-3,3,3-trifluoropropene (1233).

As a result of extensive researches, the present inventors have found that, contrary to the assumption that a carbon-fluorine bond (C—F bond) is strong and is not easy to cleave, it is possible to efficiently produce an environment-adaptive chlorofluorocarbon: 1-chloro-3,3,3-tetrafluoropropene (1233) by contact reaction of 1,3,3,3-tetrafluoropropene (1234) with hydrogen chloride in the presence of a catalyst.

The present inventors have further found that hydrogen chloride containing a small amount of hydrogen fluoride is suitably usable for the contact reaction with the 1234 in the presence of the catalyst so that, as hydrogen chloride containing unreacted hydrogen fluoride is generated as a by-product in a preceding step of producing 1,1,1,3,3-pentafluoropropane (245) from 1,1,1,3,3-pentachloropropane (240), the isomer mixture of the 1,3,3,3-tetrafluoropropene (1234) can be converted to the 1-chloro-3,3,3-trifluoropropene (1233) through the use of such by-produced hydrogen chloride.

It is thus feasible in the present invention to produce 1-chloro-3,3,3-trifluoropropene (1233) from any of trans and cis isomers of 1,3,3,3-tetrafluoropropene (1234) with the use of hydrogen chloride containing hydrogen fluoride, which is generated during production of 1,1,1,3,3-pentafluoropropane (245) or analogs thereof.

Namely, the present invention includes the following aspects 1 to 12.

[Inventive Aspect 1]

A production method of 1-chloro-3,3,3-trifluoropropene (1233), comprising bringing a raw material composition containing 1,3,3,3-tetrafluoropropene (1234) and an acid composition containing hydrogen chloride into contact with each other in gas phase in the presence of a catalyst.

[Inventive Aspect 2]

The production method according to Inventive Aspect 1, wherein the raw material composition further contains 1,1,1,3,3-pentafluoropropane (245).

[Inventive Aspect 3]

The production method according to Inventive Aspect 1 or 2, wherein the 1,3,3,3-tetrafluoropropene (1234) is obtained by fluorination of 1,1,1,3,3-pentachloropropane (240).

[Inventive Aspect 4]

The production method according to any one of Inventive Aspects 1 to 3, wherein the catalyst is a catalyst having a bond of the formula: M-X (where M is at least one kind of metal atom selected from the group consisting of aluminium (Al), titanium (Ti), iron (Fe), cobalt (Co), antimony (Sb), tin (Sn), tungsten (W), niobium (Nb), chromium (Cr) and zirconium (Zr); and X is at least one kind of halogen atom selected from the group consisting of fluorine (F), chlorine (Cl) and bromine (Br)).

[Inventive Aspect 5]

The production method according to any one of Inventive Aspects 1 to 3, wherein the catalyst is a catalyst containing a salt or oxide of at least one kind of metal selected from the group consisting of aluminium (Al), zirconium (Zr), titanium (Ti), chromium (Cr) and niobium (Nb).

[Inventive Aspect 6]

The production method according to any one of Inventive Aspects 1 to 3, wherein the catalyst is a catalyst in which a compound of at least one kind of metal selected from the group consisting of aluminium (Al), titanium (Ti), iron (Fe), cobalt (Co), antimony (Sb), tin (Sn), tungsten (W), niobium (Nb), chromium (Cr), and zirconium (Zr) is carried on carbon.

[Inventive Aspect 7]

The production method according to any one of Inventive Aspects 1 to 3, wherein the catalyst is a solid Lewis acid.

[Inventive Aspect 8]

The production method according to any one of Inventive Aspects 1 to 3, wherein the catalyst is an alumina catalyst which has been treated in advance by contact with hydrogen fluoride.

[Inventive Aspect 9]

The production method according to any one of Inventive Aspects 1 to 8, wherein the acid composition further contains hydrogen fluoride.

[Inventive Aspect 10]

The production method according to Inventive Aspect 9, wherein the amount of the hydrogen fluoride in the acid composition is 0.001 to 10 mass % based on the total amount of the hydrogen chloride and the hydrogen fluoride.

[Inventive Aspect 11]

The production method according to any one of Inventive Aspects 1 to 9, wherein the 1,3,3,3-trifluoropropene (1233) is produced from 1,1,1,3,3-pentachloropropane (240) with the use of an acid composition containing hydrogen chloride, which has been generated during production of a compound of the formula: CF₃—CH₂—CHR¹R² (where R¹ and R² are each independently a chlorine atom or a fluorine atom) or CF₃—CH═CHR³ (where R³ is a chlorine atom or a fluorine atom), as the acid composition.

[Inventive Aspect 12]

A method for producing 1,3,3,3-trifluoropropene (1233) from 1,1,1,3,3-pentachloropropane (240), comprising:

step [1] of forming a first composition containing 1,1,1,3,3-pentafluoropropane (245) and hydrogen chloride by contact of 1,1,1,3,3-pentachloropropane (240) with hydrogen fluoride;

step [2] of distilling the first composition to separate the 1,1,1,3,3-pentafluoropropane (245) and the hydrogen chloride from each other;

step [3] of forming a second composition containing 1,3,3,3-tetrafluoropropene (1234) by dehydrofluorination of the 1,1,1,3,3-pentafluoropropane (245) separated in the step [2]; and

step [4] of forming 1,3,3,3-trifluoropropene (1233) by reaction of the second composition and the hydrogen chloride separated in the step [2] in the presence of a catalyst.

It is possible by the production method of the present invention to efficiently produce the 1,3,3,3-trifluoropropene (1233) to which attention is being given as an environment-adaptive chlorofluorocarbon for a next-generation blowing agent etc. The production method of the present invention allows efficient and economical production of the 1,3,3,3-trifluoropropene (1233) with the use of hydrogen chloride containing hydrogen fluoride, which has been generated during production of the 1,1,1,3,3-pentafluoropropane (245), and with the use of any of cis isomer (1234Z) and trans isomer (1234E) which has become redundant during production of 1,3,3,3-tetrafluoropropene (1234) as the raw material.

### BRIEF DESCRIPTION OF THE DRAWINGS

FIG. 1 is a diagram showing the results of calculation of a Boltzmann distribution of trans isomer (1234E) and cis isomers (1234Z) of 1,3,3,3-tetrafluororopropene.

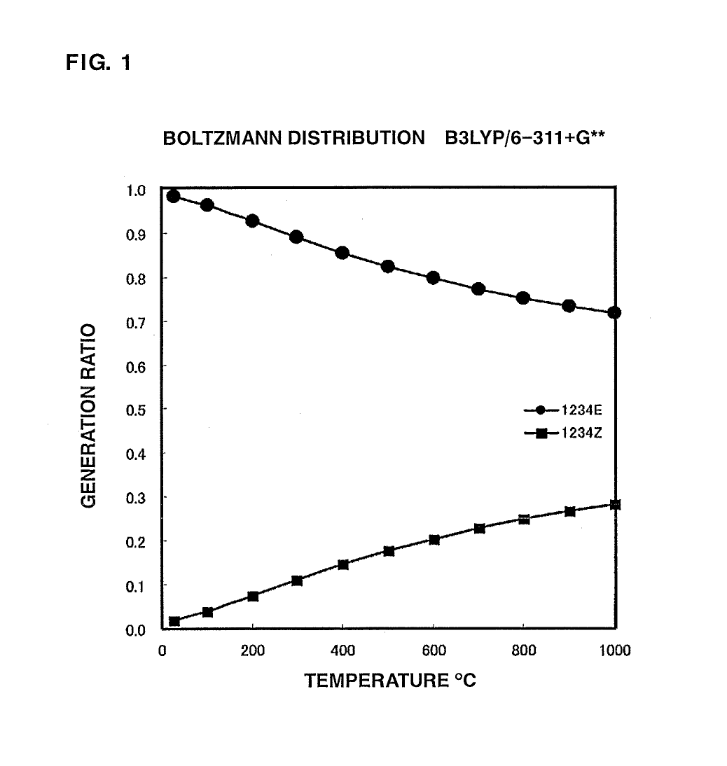

### DESCRIPTION OF THE EMBODIMENTS

Hereinafter, exemplary embodiments of the present invention will be described in detail below. It is noted that: the present invention is not limited to the following embodiments; and various changes and modifications can be made to the following embodiments based on the common knowledge of those skilled in the art without departing from the scope of the present invention.

### 1. Production Method of 1-chloro-3,3,3-trifluoropropene (1233)

A production method of 1-chloro-3,3,3-trifluoropropene (1233) according to the present invention includes bringing a raw material composition containing 1,3,3,3-tetrafluoropropene (1234) and an acid composition containing hydrogen chloride into contact with each other in gas phase in the presence of a catalyst.

The raw material composition may contain, in addition to the 1,3,3,3-tetrafluoropropene (1234), 1,1,1,3,3-pentafluoropropane (245). The acid composition may contain, in addition to the hydrogen chloride, hydrogen fluoride.

In the production method of the present invention, the 1,3,3,3-tetrafluoropropene (1234) can be produced as the raw material by any process. It is particularly efficient and economical to produce, as the raw material, the 1,3,3,3-tetrafluoropropene (1234) by dehydrofluorination of 1,1,1,3,3-pentafluoropropane (245) in the presence of a catalyst as disclosed in Patent Publication 5. It is also preferable to produce the 1,3,3,3-tetrafluoropropene (1234) by fluorination of 1,1,1,3,3-pentafluoropropane (240). After the dehydrofluorination of the 1,1,1,3,3-pentafluoropropane (245), the resulting reaction product is subjected to distillation in order to separate the trans isomer (1234E) or cis isomer (1234Z) of 1,3,3,3-tetrafluoropropene as the target compound from unreacted 1,1,1,3,3-pentafluoropropane (245). Although the 1234E can be easily separated from the 245 by distillation, the 1234Z and the 245 form an azeotropic mixture so that it is difficult to separate the 1234Z from the 245 by distillation.

There is a great demand for the low-boiling-point trans isomer (1234E, boiling point: −19° C.) as a coolant for use in an automotive air conditioner etc. On the other hand, there is a great demand for the cis isomer (1234Z, boiling point: 9° C.) as a high-temperature working fluid, a raw material for production of CF₃—C≡CH etc. It is common practice for a manufacturer to obtain an isomer mixture (1234), and then, separate the trans isomer (1234E) or cis isomer (1234Z) by precision distillation. In the production method of the present invention, the 1-chloro-3,3,3-trifluoropropene (1233) can be produced from any of the cis isomer (1234Z) and the trans isomer (1234E) which has become redundant to the manufacturer.

It is worthy of special note that the production method of the present invention allows conversion of 1,3,3,3-tetrafluoropropene (1234) containing 1,1,1,3,3-pentafluoropropane (245), that is, a mixture of the 245 and the 1234 to 1-chloro-3,3,3-trifluoropropene (1233). Namely, it feasible to use the trans isomer (1234E) or cis isomer (1234Z) of 1,3,3,3-tetrafluoropropene that contains any compound of the formula: CF₃—CH₂—CHR¹R² (where R¹ and R² are each independently a fluorine atom or a chlorine atom), such as 245, as the raw material for production of the 1-chloro-3,3,3-trifluoropropene (1233).

For example, the production method of the present invention is effective in production of the 1-chloro-3,3,3-trifluoropropene (1233) as a blowing agent from a spent mixed solvent of cis and trans isomers of 1,3,3,3-tetrafluoropropene (1234). In the production method of the present invention, the highly useful 1-chloro-3,3,3-trifluoropropene (1233) can be produced from the raw material in which the trans and cis isomers (1234E and 1234Z) of 1,3,3,3-tetrafluoropropene and the compound CF₃—CH₂—CHR¹R² are present at any ratio. It is preferable to use, as the raw material, the 1,3,3,3-tetrafluoropropene after aqueous washing and drying. A small amount of hydrogen fluoride may be contained in the 1,3,3,3-tetrafluoropropene.

Further, ultra-high purity hydrogen chloride or industrial low purity hydrogen chloride can be used as the acid composition in the production method of the present invention. In particular, hydrogen chloride containing unreacted hydrogen fluoride, which has been generated during the production of the 1,1,1,3,3-pentafluoropropane (245), is suitably usable as the acid composition. It is impossible to sell such hydrogen chloride as a product (commercial product) unless the hydrogen chloride is improved in purity by removing therefrom impurities such as hydrogen fluoride and organic substance. As there is a need to provide purification equipment such as expensive distillation column for improvement in the impurity of the hydrogen chloride, the hydrogen chloride is often disposed of as a waste after neutralization treatment. The utilization of hydrogen chloride containing hydrogen fluoride, which is generated during fluorination, is desirable for the purpose of resource conservation and waste reduction.

When hydrogen chloride containing hydrogen fluoride is generated during the fluorination or precursor preparation of the 1,1,1,3,3-pentafluoropropane (245), a C3 compound (compound of 3 carbon atoms) having a CF₃ group is also contained as an organic substance in the generated hydrogen fluoride. This C3 compound can be often converted to or separated from the 1-chloro-3,3,3-trifluoropropene (1233) and is thus suitably usable in the raw material in the production method of the present invention. Specific examples of such a compound are those of the formula: CF₃—CH₂—CHR¹R² (where R¹ and R² are each independently a chlorine atom or a fluorine atom) or CF₃—CH═CHR³ (where R³ is a chlorine atom or a fluorine atom). All of these compounds can be converted to the 1-chloro-3,3,3-trifluoropropene (1233). It is feasible to use hydrogen chloride generated in any other industrial process but is necessary to confirm that impurities contained in the hydrogen chloride can be separated from the 1-chloro-3,3,3-trifluoropropene (1233) and does not serve as a catalyst poison.

Hydrogen chloride containing even a small amount of highly corrosive and toxic hydrogen fluoride is difficult to handle and is thus often disposed of as a waste after neutralization treatment as mentioned above. It is, however, worthy of special note that the production method of the present invention allow suitable use of the hydrogen chloride containing hydrogen fluoride as the acid composition as mentioned above. The hydrogen chloride is usable as the acid composition even when the above-mentioned saturated C3 chlorofluorocarbon compound, which can be converted to the 1233, is contained in the hydrogen chloride. Further, hydrogen chloride containing a small amount of hydrogen fluoride has the effect of activating the catalyst. It is preferable to activate the catalyst by treatment with hydrogen fluoride before use in the production method of the present invention.

Roughly distilled hydrogen chloride containing hydrogen fluoride is also usable in the production method of the present invention. In the production of the 1,1,1,3,3-pentafluoropropane (245), the hydrogen chloride containing hydrogen fluoride is separated and recovered as a mixed gas. It is feasible in the production method of the present invention to use a distillation fraction of the hydrogen chloride containing hydrogen fluoride. At this time, the concentration of the hydrogen fluoride in the hydrogen chloride fraction is preferably 0.001 to 10 mass %, more preferably 0.1 to 5 mass %, based on the total amount of the hydrogen chloride fraction.

Although high-purity hydrogen chloride containing no hydrogen fluoride is usable in the production method of the present invention, it is preferable in the production method of the present invention to use the hydrogen chloride in which the hydrogen fluoride is contained in the above range because of the effect of maintaining the activity of the catalyst in the reaction. When the concentration of the hydrogen fluoride is less than 0.001 mass %, the catalyst activation effect is not obtained. There arises a possibility of damage caused to the reaction vessel when the concentration of the hydrogen fluoride exceed 10 mass %. Dry pressure distillation is one preferable technique to control the ratio of the hydrogen chloride and hydrogen fluoride.

The content ratio of the hydrogen chloride and the raw material composition in the production method of the present invention will be explained below. Herein, the raw material composition refers to a composition containing not only the trans isomer (1234E) and/or cis-isomer (1234Z) as the raw material but also the above-mentioned CF₃CH₂CHR¹R² or CF₃CHCHR³ component. The content ratio of the hydrogen chloride and the raw material composition is preferably in the range of 0.1 to 30, more preferably 0.5 to 20, in terms of mol ratio. When the mol ratio is smaller than 0.1, there may occur an unfavorable result such as insufficient conversion, caulking of the catalyst by organic substance, secular deterioration of the catalyst etc. When the mol ratio is larger than 20, the load of recovery of the hydrogen chloride becomes large.

An inert gas such as nitrogen or argon may be added when desired by one skilled in the art.

It is accordingly an preferred embodiment of the present invention to produce 1-chloro-3,3,3-trifluoropropene (1233) from 1,1,1,3,3-pentachloropropane (240) by the following steps:

step [1] of forming a first composition containing 1,1,1,3,3-pentafluoropropane (245) and hydrogen chloride by fluorination reaction of 1,1,1,3,3-pentachloropropane (240) with hydrogen fluoride;

step [2] of distilling the first composition to separate the 1,1,1,3,3-pentafluoropropane (245) and the hydrogen chloride from each other;

step [3] of forming a second composition containing 1,3,3,3-tetrafluoropropene (1234) by dehydrofluorination reaction of the 1,1,1,3,3-pentafluoropropane (245) separated in the step [2]; and

step [4] of forming 1,3,3,3-trifluoropropene (1233) by reaction of the second composition and the hydrogen chloride separated in the step [2]

### 2. Catalyst

Next, an explanation will be given of the catalyst used in the production method of the present invention.

The catalyst is preferably a catalyst containing a metal halide, a metal oxide or a metal salt as an effective component. The metal halide (M-X, M: metal, X: halogen) is a compound in which a metal atom M and a halogen atom X are bonded to each other to form a metal-halogen bond (M-X bond).

As the metal, there can be used any of metal elements of Groups 4 to 15 of the periodic table. Among others, preferred are aluminum (Al) and transition metals of atomic numbers 22 to 78 (Ti, V, Cr, Mn, Fe, Co, Ni, Cu, Zr, Nb, Mo, Tc, Ru, Rh, Pd, Ag, Hf, Ta, W, Re, Os, Ir, Pt, etc.). Specific examples of such a metal are aluminium (Al), titanium (Ti), iron (Fe), chromium (Cr), manganese (Mn), nickel (Ni), copper (Cu), cobalt (Co), zirconium (Zr), niobium (Nb), molybdenum (Mo), tin (Sn), antimony (Sb), tungsten (W), and tantalum (Ta). These metals can be used solely or in combination of two or more thereof. Magnesium, sodium or potassium may be added as a promoter.

In the metal halide, the halogen atom is either a fluorine atom, a chlorine atom, a bromine atom or an iodine atom. Among others, a fluorine atom or a chlorine atom is preferred. Particularly preferred is a fluorine atom. It is effective to positively fluorinate some or major portion of the metal surface by treatment with hydrogen fluoride etc. In the case of using a chlorofluorocarbon compound, which contains a halogen atom, as a raw material as in the production method of the present invention, a metal halide can be formed by contact of the raw material itself with the metal. It is thus feasible by using such a metal-halogen bond-formable raw material in combination with the metal to form a metal halide formed on a surface of the catalyst so that the reaction can be performed quickly under the action of the resulting catalyst.

In the metal-halogen bond-containing catalyst, the metal atom M is in a electron-deficient state as the highly electronegative halogen atom X attracts electrons of the metal atom M. In such a state, the metal often shows a Lewis acidity. The catalyst tends to show a strong Lewis acidity when the catalyst contains highly electronegative fluorine or chlorine as X or contains a large number of metal-halogen bonds. Thus, a Lewis acid catalyst is also suitably usable as the catalyst in the production method of the present invention. It can be said that the metal-halogen bond-containing catalyst where X is fluorine or chlorine or where there is included a large number of metal-halogen bonds is preferred in the present reaction.

It is preferable to use, as the catalyst in the production method of the present invention, a catalyst formed by fluorination or chlorination of the metal salt or metal oxide e.g. aluminum phosphate, aluminum sulfate, aluminum fluoride or alumina in such a manner that the catalyst has an active species of metal halide containing an Al—F bond etc. on a surface thereof. In particular, a catalyst prepared by molding of aluminum fluoride powder has an Al—F bond-containing active species on a surface thereof and thus can suitably be used as it is as the catalyst in the production method of the present invention. It is further preferable to perform activation treatment on the catalyst by contact with hydrogen fluoride and intentionally form an Al—F bond-containing active species on a surface of the catalyst.

The presence or absence of M-X bond can be verified by instrumental analysis such as EXAFS, XAFS, XRF, XPS, IR or XRD. One example of M-X bond verification technique is to remove any physically adsorbed halogen atom from the surface of the catalyst by firing at 300° C. after the completion of the reaction, and then, detect the metal atom and the halogen atom by the above analytical means. The firing time required for removal of the physically adsorbed halogen atom is preferably 12 to 120 hours. The physically adsorbed halogen atom can be removed more efficiently by firing under the flow of nitrogen or under reduced pressure. Particularly preferred are XAFS and XPS, each of which enables detailed analysis of the state of metal-halogen bond. In general, the physically adsorbed halogen atom is present in the form of a hydrogen halide.

The size (maximum particle diameter) of the catalyst is preferably 1/30 to ⅓, more preferably 1/20 to ⅕, of the inner diameter of the reaction vessel used in the reaction. When the size of the catalyst is larger than the above range, the raw material may pass through the reaction vessel without contacting the catalyst. There arises a possibility of increase in the pressure loss of the reaction vessel or clogging of the reaction vessel when the size of the catalyst is smaller than the above range. More specifically, the size of the catalyst is preferably in the range of 0.5 to 20 mm, more preferably 2 to 10 mm.

The catalyst can be prepared in the above size by molding the oxide or salt of any of metals of atomic numbers 13 to 78 into spherical form or pellet form. Although one skilled in the art may prepare the catalyst by pressure-molding the metal oxide or metal salt with the use of a compression molding machine, it is feasible to use commercially available pellets or balls (spheres) composed predominantly of alumina (γ-alumina, α-alumina etc.), titania zirconia as the catalyst. Alternatively, the catalyst can be provided in the form of an impregnated catalyst by using activated carbon (coconut shell coal, charcoal, peat coal etc.) or any of the above metal oxides as a carrier and impregnating the carrier with a solution of the above-mentioned effective metal component. It is preferable to subject, in advance, the carrier to fluorination treatment by hydrogen fluoride etc.

There is no particular limitation on the preparation process of the impregnated catalyst. The impregnated catalyst can be prepared by provided a solution of a soluble compound of the metal, such as nitrate, chloride or oxyhalide, impregnating the carrier with the solution or spraying the solution onto the carrier, drying the solution-applied carrier, and then, bringing the resulting metal salt-carrying carrier into contact with hydrogen fluoride, hydrogen chloride, chlorofluorohydrocarbon etc. under heating for fluorination of part or the whole of the carried metal or the carrier. A fluorination product of alumina, titania, stainless steel etc. (such as fluorinated alumina) and activated carbon are also usable as the catalyst. There is no particular limitation on the fluorination process. For example, the fluorinated alumina catalyst can be prepared by providing alumina commercially available for drying use or use as a catalyst carrier, flowing hydrogen fluoride to the alumina while heating the alumina, and thereby bringing the alumina into contact with hydrogen fluoride in gas phase, or by providing an aqueous solution of hydrogen fluoride, spraying the solution to the alumina or immersing the alumina in the solution at around room temperature, and then, drying the solution-applied alumina.

In the case where the metal compound is liquid at around room temperature, such as antimony pentachloride, tin tetrachloride or titanium tetrachloride, the catalyst may be prepared by impregnating the metal compound as it is into the activated carbon, alumina etc.

Regardless of whether the catalyst is prepared by any process, it is preferable to active the catalyst by contact with hydrogen fluoride or another fluorinating agent such as fluorine-containing hydrocarbon before use.

Particularly preferred examples of the catalyst are alumina, titania, zirconia, chromium/activated carbon, nickel/activated carbon, iron/activated carbon, antimony/activated carbon, chromium/alumina and chromium/zirconia. It is a preferred embodiment of the present invention to, before the reaction, perform surface treatment on the above catalyst by contact with hydrogen fluoride and thereby form a metal-fluorine bond(s) on the catalyst. The thus-obtained catalyst shows high catalytic activity and thus can suitably be used in the present reaction.

### 3. Reaction Conditions

The reaction conditions of the production method of the present invention will be next explained below.

The reaction system is preferably a gas-phase flow system in the production method of the present invention. Specific examples of the reaction system are a fixed-bed flow system, a fixed-bed circulation system, a fluidized-bed flow system or the like. Among others, it is convenient to adopt a fixed-bed gas-phase flow system. Further, it is convenient to perform the reaction at around normal pressure (i.e. under atmospheric pressure conditions) although the reaction can be performed under reduced pressure conditions or under pressurized conditions.

In the production method of the present invention, the reaction temperature is varied depending on the kind and state of the catalyst, the contact time etc. and is preferably in the range of 200 to 500° C., more preferably 250 to 400° C. When the reaction temperature is lower than 200° C., the conversion rate may be lowered. There may occur an unfavorable result such as increase in side reaction, caulking etc. when the reaction temperature is higher than 500° C.

The contact time is varied depending on the kind of the raw material composition etc. and is generally in the range of 0.1 to 200 seconds, preferably 2 to 150 seconds. When the contact time is shorter than 0.1 second, there arises a possibility of low conversion from the 1,3,3,3-tetrafluoropropene (1234) to the 1-chloro-3,3,3-trifluoropropene (1233) or increase in the pressure loss of the reaction vessel.

In the case where the catalytic activity of the catalyst is deteriorated during long-time operation, it is feasible to remove caulking substance from the surface of the catalyst by contact oxidation with air or chlorine under temperature conditions of 250 to 800° C. At this time, there may occur sudden generation of heat when the oxidation treatment is performed with the use of 100% air or chlorine. It is thus preferable to perform the oxidation treatment while diluting such oxidation gas with an inert gas e.g. nitrogen for safety purposes. It is also preferable in the oxidation treatment to control the rate of dilution of the oxidation gas with the inert gas by checking the temperature of the heat spot in the reaction vessel.

In the gas-phase flow system, the product can be easily recovered and collected by cooling. The cooling temperature is preferably −80 to 5° C. There are not only a need to provide special refrigerator equipment, but also a possibility of solidification of the product, when the cooling temperature is lower than −80° C. The collection efficiency may be lowered when the cooling temperature is higher than 5° C. It is preferable to remove fluorine ions or chlorine ions from the collected product by washing with water or an aqueous basic solution. The washing operation can be performed in a batch process or a continuous process. There may occur heat of neutralization when the washing operation is performed with the use of the aqueous alkaline solution. It is thus recommendable to first wash away the major portion of the chlorine or fluorine ions by water from the product, wash the product with the aqueous alkaline solution, and then, wash away the alkaline component by water from the product. Further, it is preferable to dry the washed product with a solid dehydrating agent such as zeolite.

In view of the fact that most organic substances get decomposed in a temperature range of 200 to 300° C., there is a possibility that high-temperature reaction such as that of the present invention may cause decomposition or unexpected reaction of raw material or reaction product and thereby generate any impurity by-product difficult to separate by distillation purification. As high purity is required for use of the 1-chloro-3,3,3-trifluoropropene as a working fluid, a cleaning agent, a solvent, a blowing agent etc., the generation of the difficult-to-separate by-product in the reaction should be avoided even when the reaction is high in yield. In the production method of the present invention, the 1-chloro-3,3,3-trifluoropropene (1233) can be easily produced at a high purity, with substantially no generation of any material difficult to separate by distillation, under the above-mentioned preferable reaction conditions.

There is no particular limitation on the distillation column used in the production method of the present invention. The theoretical plate number of the distillation column is preferably 10 to 30. When the theoretical plate number is less than 10, the distillation yield may be low. The distillation purification can be performed with no particular problem when the theoretical plate number exceeds 30. In this case, however, the distillation column may become high in equipment cost and running cost. As the trans isomer (1233E) has a boiling point of 19° C., the coolant temperature of the distillation column is generally −50 to 5° C. When the coolant temperature is lower than −50° C., the distillation column needs to be provided with special cooling equipment and becomes high in running cost. When the coolant temperature is higher than 5° C., there increases a distillation loss unless the distillation operation is performed under pressurized conditions. The coolant temperature is preferably −20 to 0° C.

For use of the 1-chloro-3,3,3-trifluoropropene as a working fluid, a cleaning agent, a solvent, a blowing agent etc., it is necessary to control not only the organic purity but also the halogen ion concentration and moisture content of the 1-chloro-3,3,3-trifluoropropene. In the case of using the 1-chloro-3,3,3-trifluoropropene as a working fluid for any equipment, the halogen ion such as fluorine ion or chlorine ion or the moisture content may become a cause of corrosion of the equipment. In the case of using the 1-chloro-3,3,3-trifluoropropene as a cleaning agent for metal parts, the halogen ion or the moisture content may become a cause of corrosion of the metal parts. In the case of using the 1-chloro-3,3,3-trifluoropropene as a blowing agent, the halogen ion or the moisture content becomes a cause of poisoning by reaction with an amine catalyst. In other words, the 1-chloro-3,3,3-trifluoropropene is a product of quality suitable for use as a working fluid, a cleaning agent, a solvent, a blowing agent etc. when the organic purity, halogen ion concentration and moisture content of the 1-chloro-3,3,3-trifluoropropene are in their respective preferable ranges. In general, the organic purity is preferably 99% or higher, more preferably 99.5% or higher. The moisture content is preferably 100 ppm or less, more preferably 30 ppm or less. The acidic component concentration is preferably 5 ppm or less, more preferably 0.5 ppm or less.

### EXAMPLES

The present invention will be described in more detail below by way of the following examples. It is noted that the following examples are illustrative and are not intended to limit the present invention thereto. Herein, the unit “%” of each of composition analysis values means the area percentage “area %” of an individual component as determined by gas chromatography (detector: FID) of a reaction mixture. Further, the composition analysis values were rounded off to the respective indicated numbers. For example, the value “0.00%” in the following tables means to be less than 0.005 area %.

### Preparation Example 1

### Catalyst Preparation

Provided was a reaction vessel of stainless steel (SUS316) having a length of 240 mm and an inner diameter of 3/4 inch. This reaction vessel was packed with 36 g of γ-alumina beads (product name: KHS-46 manufactured by Sumika Alchem Co., Ltd.) as a catalyst, followed by controlling the jacket temperature of the reaction vessel to be 50° C. while flowing nitrogen at a rate of 50 cc/min into the reaction vessel, and then, flowing hydrogen fluoride at a rate of 0.4 g/min into the reaction vessel through a carburetor. By adsorption of the hydrogen fluoride on the alumina, the generation of heat of adsorption and heat of reaction was observed, in particular, in the vicinity of the inlet of the reaction vessel. The heat generation area was gradually shifted toward the outlet of the reaction vessel. This local heat generation was retarded by decreasing the flow rate of the hydrogen fluoride to 0.1 g/min when the highest-temperature heat spot exceeded 400° C. When the heat generation area reached the outlet of the reaction vessel, the jacket temperature of the reaction vessel was raised by 50° C. up to 350° C. The same operation as above was repeated. After that, the jacket temperature of the reaction vessel was maintained at 350° C. The flow rate of the hydrogen fluoride was gradually increased to 0.7 g/min. As in the case of the above treatment operation, the flow rate of the hydrogen fluoride was decreased to 0.1 g/min when the highest-temperature heat spot exceeded 400° C. When the heat spot was substantially no longer observed under the conditions of the jacket temperature of 350° C. and the hydrogen fluoride flow rate of 0.4 g/mm, the activation treatment of the catalyst was continued under the same conditions for 2 hours. The reaction vessel was cooled down by turning off the heater while keeping the flow of the nitrogen. There was thus obtained the reaction vessel in which the alumina catalyst activated by contact treatment with the hydrogen fluoride was packed.

### Example 1

### Raw Material: Trans Isomer (1234E)

The above reaction vessel packed with the alumina catalyst was heated in an electric furnace under the flow of nitrogen at a rate of 15 ml/min. When the reaction vessel and the catalyst reached a temperature of 350° C., hydrogen chloride containing 2.3 mass % of hydrogen fluoride was introduced at a rate of 166 ml/min into the reaction vessel through the carburetor. Further provided was a 1000-ml cylinder filled with trans isomer (1234E, 99.9 GC %, see TABLE 1 for the detailed component ratio of the raw material composition). While keeping the flow of the hydrogen chloride, the trans isomer was introduced at a rate of 0.14 g/min (27.5 ml/min) from the cylinder into the reaction vessel through the carburetor. After confirming that the temperature of the reaction vessel became steady at 360° C., the flow of the nitrogen was stopped. The outlet gas from the reaction vessel was sampled and subjected to composition analysis as follows. The outlet gas was collected in an polyethylene bag of 100 cc capacity with water (10 g) put therein. The bag was shaken to absorb an acidic component of the outlet gas into the water. The bag was subsequently heated at 50° C. in an oven. The resulting gas phase was analyzed by a chromatograph with a FID detector. The reaction conditions are shown in TABLE 1; and the reaction results are shown in TABLE 2.

### Examples 2 to 4

The conversion reaction of trans isomer (1234E) to 1-chloro-3,3,3-trifluoropropene (1233) was performed in the same manner as in Example 1, under the reaction conditions shown in TABLE 1, except for using hydrogen chloride of purity 99.99%. The reaction conditions are shown in TABLE 1; and the reaction results are shown in TABLE 2.

As is seen from the results of Examples 1 to 4 in TABLE 2, it was possible by the production method of the present invention to produce the 1-chloro-3,3,3-trifluoropropene (1233) at a high yield.

### Examples 5 to 9

### Raw Material: Cis Isomer (1234Z)

Using cis isomer (1234Z) as a raw material, the conversion reaction of cis isomer (1234Z) to 1-chloro-3,3,3-trifluoropropene (1233) was performed in the same manner as in Example 1 under the reaction conditions shown in TABLE 1. Herein, hydrogen chloride of purity 99.99% was used in Examples 5 and 7 to 9; and hydrogen chloride containing hydrogen fluoride was used in Example 6 as in Example 1. The reaction conditions are shown in TABLE 1; and the reaction results are shown in TABLE 2.

As is seen from the results of Examples 5 to 9 in TABLE 2, it was possible by the production method of the present invention to produce the 1-chloro-3,3,3-trifluoropropene (1233) at a high yield, as in the case of Examples 1 to 4, even when the cis isomer (1234Z) was used as the raw material.

TABLE 1

| | Reaction | HCl | Raw material | HCl/raw | | |
|---|---|---|---|---|---|---|
| | temperature | flow rate | flow rate | material | Contact | |
| Ex. | (° C.) | (cc/min) | (cc/min) | mol ratio | time (s) | HCl |
| 1 | 360 | 166 | 27.5 | 6.0 | 5.6 | HCl containing |
| | | | | | | HF (2.3%) |
| 2 | 360 | 78 | 27.5 | 2.8 | 9.8 | High-purity HCl |
| 3 | 280 | 214 | 27.5 | 7.8 | 5.9 | High-purity HCl |
| 4 | 280 | 43 | 27.5 | 1.6 | 20.3 | High-purity HCl |
| 5 | 300 | 179 | 27.5 | 6.5 | 4.9 | High-purity HCl |
| 6 | 310 | 86 | 27.5 | 3.1 | 4.8 | HCl containing |
| | | | | | | HF (2.3%) |
| 7 | 350 | 84 | 27.5 | 3.1 | 4.5 | High-purity HCl |
| 8 | 350 | 87 | 27.5 | 3.1 | 2.2 | High-purity HCl |
| 9 | 350 | 44 | 27.5 | 1.6 | 4.5 | High-purity HCl |

TABLE 2

| | Conversion | 1233E + Z | GC area % | | | | | | | | |
|---|---|---|---|---|---|---|---|---|---|---|---|
| Ex. | rate (%) | yield (%) | TFPy | 1234E | 1234zc | 245fa | 1234Z | 1233E | 244fa | 1233Z | others |
| Raw material A | | | 0.00 | 99.93 | 0.00 | 0.03 | 0.04 | 0.00 | 0.00 | 0.00 | 0.00 |
| 1 | 99.7 | 95.3 | 0.04 | 1.56 | 0.00 | 0.11 | 0.30 | 86.08 | 0.05 | 9.25 | 2.60 |
| 2 | 99.6 | 95.8 | 0.04 | 1.74 | 0.00 | 0.30 | 0.36 | 85.64 | 0.11 | 10.18 | 1.64 |
| 3 | 92.1 | 45.2 | 0.00 | 42.84 | 0.01 | 3.04 | 7.86 | 40.94 | 0.04 | 4.21 | 1.06 |
| 4 | 95.4 | 60.7 | 0.01 | 23.86 | 0.00 | 10.25 | 4.56 | 54.84 | 0.18 | 5.84 | 0.44 |
| Raw material B | | | 0.00 | 0.00 | 0.00 | 0.14 | 99.59 | 0.23 | 0.00 | 0.00 | 0.04 |
| 5 | 99.7 | 96.6 | 0.00 | 1.39 | 0.00 | 0.25 | 0.26 | 87.71 | 0.09 | 8.89 | 1.40 |
| 6 | 99.4 | 94.8 | 0.00 | 2.62 | 0.00 | 0.47 | 0.55 | 84.44 | 0.11 | 10.37 | 1.44 |
| 7 | 99.7 | 96.1 | 0.01 | 1.43 | 0.00 | 0.15 | 0.30 | 85.73 | 0.07 | 10.33 | 1.98 |
| 8 | 97.9 | 85.6 | 0.01 | 8.83 | 0.01 | 0.21 | 2.09 | 75.18 | 0.02 | 10.38 | 3.29 |
| 9 | 98.3 | 88.7 | 0.01 | 7.65 | 0.00 | 0.30 | 1.71 | 78.40 | 0.03 | 10.30 | 1.60 |

TFPy (CF₃—CH≡CH),
1234E (trans-CF₃CH═CHF),
1234zc (CHF₂CH═CF₂),
245fa (CF₃CH₂CHF₂),
1234Z (cis-CF₃CH═CHF),
1233E (trans-CF₃CH═CHCl),
244fa (CF₃CH₂CHClF),
1233Z (cis-CF₃CH═CHCl)

### Example 10

Under the same reaction conditions as those of Example 5, the reaction was continued for about 100 hours. The resulting product gas was collected in a stainless cylinder cooled with dry ice, washed with ice water and dried with zeolite, thereby yielding 790 g of crude product. The crude product was distilled into fractions by a glass distillation column with a theoretical plate number of 20. The distillation fraction of 99% purity trans isomer was provided to Example 11. The distillation fraction between 38 to 41° C., containing cis isomer (1233Z) as a main component, was provided to Example 12.

### Example 11

First, 310 g of the distillation fraction of 99% purity trans isomer (1233E) obtained in Example 10 was stored in a refrigerator of 10° C. Then, 100 g of the distillation fraction was sampled and put into an ultrasonic cleaning system. In this cleaning system, a glass lens with fingerprints was subjected to cleaning for 100 seconds. After the cleaning, the glass lens was dried for 60 seconds by a drier. It was confirmed by visual observation of the glass lens that the fingerprints were cleaned off.

### Example 12

The same experiment as that of Example 11 was performed using 36 g of the distillation fraction (main component: cis isomer (1233Z)) obtained between 38 to 41° C. in Example 10. It was confirmed that the fingerprints were cleaned off as in the case of Example 11.

As is seen from the results of Examples 10 to 12, it was possible that the 1-chloro-3,3,3-trifluoropropene (1233) produced by the production method of the present invention could be purified by distillation to a level suitable for use as a cleaning agent etc.

### Example 13

First, 45 g of zirconium oxychloride (ZrOCl₂.8H₂O) and 5 g of niobium pentachloride (NbCl₅) were dissolved in 400 ml of ethanol. In this solution, 500 ml of particulate γ-alumina of 5 mm in diameter (product name: KHS-46 manufactured by Sumika Alchem Co., Ltd.) was immersed and left for one day. After that, the ethanol was evaporated by a rotary evaporator. A catalyst was obtained by drying the evaporation residue at 150° C. under reduced pressure, and then, subjecting the dried residue to fluorination treatment in the same manner as in Preparation Example 1. Using the thus-obtained catalyst, the same reaction experiment as that of Example 6 was performed. The reaction conditions are shown in TABLE 3; and the reaction results are shown in TABLE 4.

### Example 14

The same reaction experiment as that of Example 13 was performed using particulate chromia (product name: E01 W-1 manufactured by JGC Catalysts&Chemicals Ltd.). Herein, the particulate chromia was prepared as a catalyst by fluorination treatment in the same manner as in Preparation Example 1. The reaction conditions are shown in TABLE 3; and the reaction results are shown in TABLE 4.

As is seen from the results of Examples 13 and 14 in TABLE 4, it was possible by the production method of the present invention to produce 1-chloro-3,3,3-trifluoropropene (1233) with high yield, as in the case of Examples 1 to 9, even when the kind of the catalyst was changed.

TABLE 3

| | | Reaction | HCl | Raw material | HCl/raw | | |
|---|---|---|---|---|---|---|---|
| | | temperature | flow rate | flow rate | material | Contact | |
| Ex. | Catalyst | (° C.) | (cc/min) | (cc/min) | mol ratio | time (s) | HCl |
| 13 | Zr—Nb/ | 310 | 86 | 27.5 | 3.1 | 4.8 | HCl containing |
| | alumina | | | | | | HF (2.3%) |
| 14 | Particulate | 310 | 86 | 27.5 | 3.1 | 4.8 | HCl containing |
| | chromia | | | | | | HF (2.3%) |

TABLE 2

| | Conversion | 1233E + Z | GC area % | | | | | | | | |
|---|---|---|---|---|---|---|---|---|---|---|---|
| Ex. | rate (%) | yield (%) | TFPy | 1234E | 1234zc | 245fa | 1234Z | 1233E | 244fa | 1233Z | others |
| Raw material | | | 0.00 | 0.00 | 0.00 | 0.14 | 99.59 | 0.23 | 0.00 | 0.00 | 0.04 |
| 13 | 99.5 | 95.0 | 0.00 | 2.61 | 0.00 | 0.40 | 0.52 | 84.59 | 0.09 | 10.38 | 1.41 |
| 14 | 99.8 | 94.3 | 0.00 | 2.62 | 0.00 | 0.51 | 0.19 | 83.98 | 0.12 | 10.31 | 2.27 |

TFPy (CF₃—CH≡CH),
1234E (trans-CF₃CH═CHF),
1234zc (CHF₂CH═CF₂),
245fa (CF₃CH₂CHF₂),
1234Z (cis-CF₃CH═CHF),
1233E (trans-CF₃CH═CHCl),
244fa (CF₃CH₂CHClF),
1233Z (cis-CF₃CH═CHCl)

1. A production method of 1-chloro-3,3,3-trifluoropropene, comprising bringing a raw material composition containing 1,3,3,3-tetrafluoropropene and an acid composition containing hydrogen chloride into contact with each other in gas phase in the presence of a catalyst.

2. The production method according to claim 1, wherein the raw material composition further contains 1,1,1,3,3-pentafluoropropane.

3. The production method according to claim 1, wherein the 1,3,3,3-tetrafluoropropene is obtained by fluorination of 1,1,1,3,3-pentachloropropane.

4. The production method according to claim 1, wherein the catalyst is a catalyst having a bond of the formula: M-X where M is at least one kind of metal atom selected from the group consisting of aluminium (Al), titanium (Ti), iron (Fe), cobalt (Co), antimony (Sb), tin (Sn), tungsten (W), niobium (Nb), chromium (Cr), and zirconium (Zr); and X is at least one kind of halogen atom selected from the group consisting of fluorine (F), chlorine (Cl) and bromine (Br)).

5. The production method according to claim 1, wherein the catalyst is a catalyst containing a salt or oxide of at least one kind of metal selected from the group consisting of aluminium (Al), zirconium (Zr), titanium (Ti), chromium (Cr) and niobium (Nb).

6. The production method according to claim 1, wherein the catalyst is a catalyst in which a compound of at least one kind of metal selected from the group consisting of aluminium (Al), titanium (Ti), iron (Fe), cobalt (Co), antimony (Sb), tin (Sn), tungsten (W), chromium (Cr), niobium (Nb) and zirconium (Zr) is carried on carbon.

7. The production method according to claim 1, wherein the catalyst is a solid Lewis acid.

8. The production method according to claim 1, wherein the catalyst is an alumina catalyst which has been treated in advance by contact with hydrogen fluoride.

9. The production method according to claim 1, wherein the acid composition further contains hydrogen fluoride.

10. The production method according to claim 9, wherein the amount of the hydrogen fluoride in the acid composition is 0.001 to 10 mass % based on the total amount of the hydrogen chloride and the hydrogen fluoride.

11. The production method according to claim 1, wherein the 1,3,3,3-trifluoropropene is produced from 1,1,1,3,3-pentachloropropane by using an acid composition containing hydrogen chloride, which has been generated during production of a compound of the formula: CF₃—CH₂—CHR¹R² (where R¹ and R² are each independently a chlorine atom or a fluorine atom) or CF₃—CH═CHR³ (where R³ is a chlorine atom or a fluorine atom), as the acid composition.

12. A method for producing 1,3,3,3-trifluoropropene from 1,1,1,3,3-pentachloropropane, comprising:
step [1] of forming a first composition containing 1,1,1,3,3-pentafluoropropane and hydrogen chloride by contact of 1,1,1,3,3-pentachloropropane with hydrogen fluoride;
step [2] of distilling the first composition to separate the 1,1,1,3,3-pentafluoropropane and the hydrogen chloride from each other;
step [3] of forming a second composition containing 1,3,3,3-tetrafluoropropene by dehydrofluorination of the 1,1,1,3,3-pentafluoropropane separated in the step [2]; and
step [4] of forming 1,3,3,3-trifluoropropene by reaction of the second composition and the hydrogen chloride separated in the step [2] in the presence of a catalyst.
